(19) United States
(12) Patent Application Publication
(10) Pub. No.: US 2019/0046263 A1
(43) Pub. Date: Feb. 14, 2019

(54) TREATMENT SYSTEM AND CONTROL DEVICE

(71) Applicant: OLYMPUS CORPORATION, Tokyo (JP)

(72) Inventors: Tsuyoshi HAYASHIDA, Hachioji-shi (JP); Satomi SAKAO, Hachioji-shi (JP)

(73) Assignee: OLYMPUS CORPORATION, Tokyo (JP)

(21) Appl. No.: 16/164,996

(22) Filed: Oct. 19, 2018

Related U.S. Application Data
(63) Continuation of application No. PCT/JP2016/063090, filed on Apr. 26, 2016.

(51) Int. Cl. A61B 18/14

(52) U.S. Cl. CPC A61B 18/1445; A61B 2018/00589

(57) ABSTRACT

In a treatment system, a treatment instrument includes a pair of grasping pieces each of which includes an electrode. A control device outputs electric energy to the electrodes and thereby supplies a high-frequency current to a treatment target. The control device switches between a first mode and a second mode that is different from the first mode in control scheme regarding output of the electric energy to the electrodes, based on a load that acts on one of the grasping pieces.

This is a Continuation Application of PCT Application No. PCT/JP2016/063090, filed Apr. 26, 2016, the entire contents of which are incorporated herein by reference.

### BACKGROUND

Exemplary embodiments relate to a treatment system including an energy treatment instrument which applies treatment energy to a treatment target grasped between a pair of grasping pieces, and relates to a control device for use in the treatment system.

PCT International Publication No. 2012/061638 discloses an energy treatment instrument which grasps a treatment target, such as a biological tissue, between a pair of grasping pieces. In this energy treatment instrument, the grasping pieces are respectively provided with electrodes. When electric energy is supplied to both electrodes, a high-frequency current flows between the electrodes through the grasped treatment target. The high-frequency current is thereby applied as treatment energy to the treatment target.

### SUMMARY

According to at least one exemplary embodiment, a treatment system comprises a treatment instrument including a first grasping piece, a second grasping piece, and a sensor, the first grasping piece including a first electrode, the second grasping piece including a second electrode that is different from the first electrode, the second grasping piece being configured to grasp a treatment target together with the first grasping piece by opening and closing with respect to the first grasping piece, and the sensor being configured to detect a load that acts on the second grasping piece; and a control device configured to output electric energy to the first electrode and the second electrode and thereby configured to supply a high-frequency current to the treatment target for treating the treatment target, the control device being further configured to switch between a first mode and a second mode that is different from the first mode in control scheme regarding output of the electric energy to the first electrode and the second electrode, based on the load detected by the sensor.

According to another exemplary embodiment, a control device is configured to be used with a treatment instrument, the treatment instrument including a first grasping piece, a second grasping piece, and a sensor, the first grasping piece including a first electrode, the second grasping piece including a second electrode that is different from the first electrode, the second grasping piece being configured to grasp a treatment target together with the first grasping piece by opening and closing with respect to the first grasping piece, and the sensor being configured to detect a load acting on the second grasping piece, the control device being configured to: obtain the load detected by the sensor; output electric energy to the first electrode and the second electrode and thereby supply a high-frequency current to the treatment target for treating the treatment target; and switch between a first mode and a second mode based on the load detected by the sensor, the second mode being different from the first mode in control scheme regarding output of the electric energy to the first electrode and the second electrode.

Advantages will be set forth in the description which follows, and in part will be apparent from the description, or may be learned by practice of exemplary embodiments. The advantages may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate exemplary embodiments.

FIG. 1 is a schematic diagram illustrating a treatment system according to an exemplary embodiment;

FIG. 2 is a block diagram illustrating a control configuration in the treatment system according to an exemplary embodiment;

FIG. 3 is a schematic diagram showing a sensor according to an example of an exemplary embodiment;

FIG. 4 is a schematic diagram showing a sensor according to an exemplary embodiment;

FIG. 5 is a flowchart illustrating a process executed by the processor in a seal treatment of a blood vessel using the treatment system according an exemplary embodiment; FIG. 6 is a flowchart illustrating a process in output control in a first seal mode of the processor according to an exemplary embodiment;

FIG. 7 is a schematic diagram illustrating an example of a variation with time of an impedance between a pair of grasping pieces, in a state in which the processor according to an exemplary embodiment is executing output control in the first seal mode and in the second seal mode;

FIG. 8 is a schematic diagram illustrating a state in which a blood vessel is grasped between the grasping pieces without being pulled, according to an exemplary embodiment;

FIG. 9 is a schematic diagram illustrating a state of the blood vessel being grasped between the grasping pieces and pulled to one side in a direction intersecting with an extending direction of the blood vessel according to an exemplary embodiment;

FIG. 10 is a schematic diagram illustrating an example of a variation with time of an impedance between the pair of grasping pieces, in a state in which the processor according to an exemplary embodiment is executing the output control in the first seal mode and in the second seal mode;

FIG. 11 is a flowchart illustrating a process in the second seal mode of the output control executed by the processor according to an exemplary embodiment;

FIG. 12 is a schematic diagram illustrating an example of a variation with time of an impedance between the pair of grasping pieces, in a state in which the processor according to an exemplary embodiment is executing the output control in the first seal mode and in the second seal mode;

FIG. 13 is a flowchart illustrating a process executed in the second seal mode of the output control by the processor according to an exemplary embodiment;

FIG. 14 is a flowchart illustrating a process executed in the seal treatment of the blood vessel by the processor using the treatment system according to an exemplary embodiment;

FIG. 15 is a block diagram illustrating a control configuration in a treatment system according to an exemplary embodiment;

FIG. 16 is a schematic view illustrating an example of a grasping force adjustment element according to an exemplary embodiment; and

FIG. 17 is a flowchart illustrating a process executed in the seal treatment of the blood vessel by the processor using the treatment system according to an exemplary embodiment.

### DETAILED DESCRIPTION

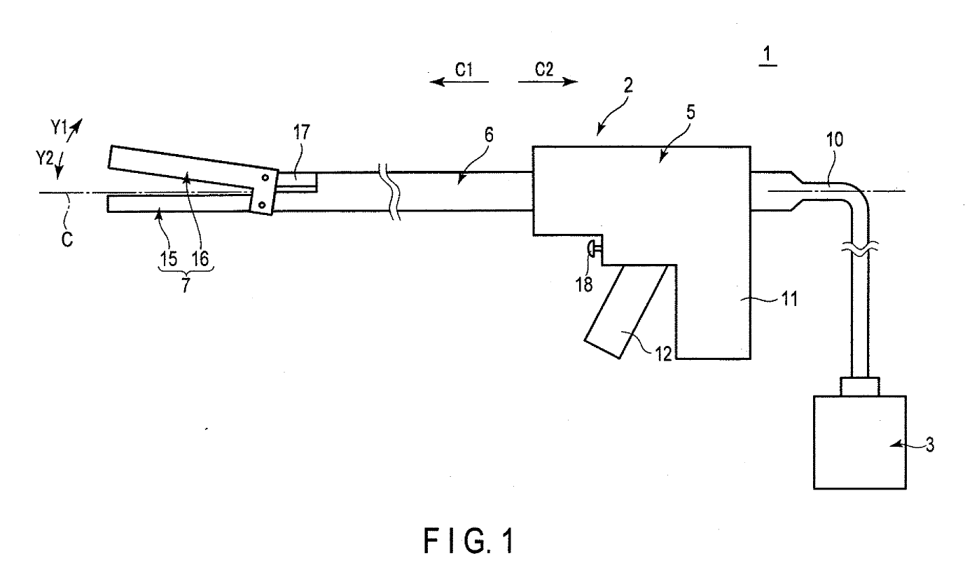

An exemplary embodiment will be described with reference to FIGS. 1 to 9. FIG. 1 is a view illustrating a treatment system 1 according to the present embodiment. As illustrated in FIG. 1, the treatment system 1 includes an energy treatment instrument 2 and a control device (energy control device) 3. The energy treatment instrument 2 has a longitudinal axis C. Here, one side of a direction along the longitudinal axis C is defined as a distal side (arrow C1 side), and the side opposite to the distal side is defined as a proximal side (arrow C2 side).

The energy treatment instrument 2 includes a housing 5 which can be hand-held, a sheath (shaft) 6 coupled to the distal side of the housing 5, and an end effector 7 provided in a distal portion of the sheath 6. One end of a cable 10 is connected to the housing 5 of the energy treatment instrument 2. The other end of the cable 10 is detachably connected to the control device 3. The housing 5 is provided with a grip (stationary handle) 11, and a handle (movable handle) 12 is rotatably attached to the housing 5. In accordance with the handle 12 rotating relative to the housing 5, the handle 12 opens or closes relative to the grip 11. According to the present embodiment, the handle 12 is located on the distal side with respect to the grip 11, and the handle 12 moves substantially in parallel to the longitudinal axis C in the opening or closing motion relative to the grip 11. The embodiment, however, is not limited thereto. In one example, the handle 12 may be located on the proximal side with respect to the grip 11. In another example, the handle 12 may be located on the side opposite to the grip 11 with respect to the longitudinal axis C, and a moving direction in the opening or closing motion relative to the grip 11 may intersect with the longitudinal axis C (may be substantially perpendicular to the longitudinal axis C).

The sheath 6 extends along the longitudinal axis C. The end effector 7 includes a first grasping piece 15, and a second grasping piece 16 which is configured to open and close relative to the first grasping piece 15. The handle 12 and the end effector 7 are coupled via a movable member 17 that extends inside the sheath 6 along the longitudinal axis C. By opening or closing the handle 12, which is an opening and closing operation input section, relative to the grip 11, the movable member 17 moves along the longitudinal axis C relative to the sheath 6 and housing 5, thereby opening or closing the pair of grasping pieces 15 and 16 relative to each other. When the grasping pieces 15 and 16 are closed relative to each other, the grasping pieces 15 and 16 grasp a biological tissue, such as a blood vessel, as a treatment target. The opening and closing directions (directions of arrow Y1 and arrow Y2) of the grasping pieces 15 and 16 intersect the longitudinal axis C (i.e., they are substantially perpendicular to the longitudinal axis C).

The end effector 7 will suffice as long as the paired grasping pieces 15 and 16 is configured to open or close relative to each other in accordance with the opening or closing motion of the handle 12. In one example, one of the grasping pieces 15 and 16 is formed integrally with the sheath 6 or fixed to the sheath 6, while the other one of the grasping pieces 15 and 16 is pivotally attached to the distal portion of the sheath 6. In another example, both of the grasping pieces 15 and 16 are pivotally attached to the distal portion of the sheath 6. In still another example, a rod member (not shown) is inserted through the sheath 6, and a portion of the rod member (probe) projecting from the sheath 6 toward the distal side forms one of the grasping pieces 15 and 16. The other one of the grasping pieces 15 and 16 is pivotally attached to the distal portion of the sheath 6. In still another example, a rotary knob (not shown) may be attached to the housing 5. If this is the case, by turning the rotary knob around the longitudinal axis C relative to the housing 5, the sheath 6 and the end effector 7 turn together with the rotary knob around the longitudinal axis C relative to the housing 5. In this manner, the angular position of the end effector 7 around the longitudinal axis C can be adjusted.

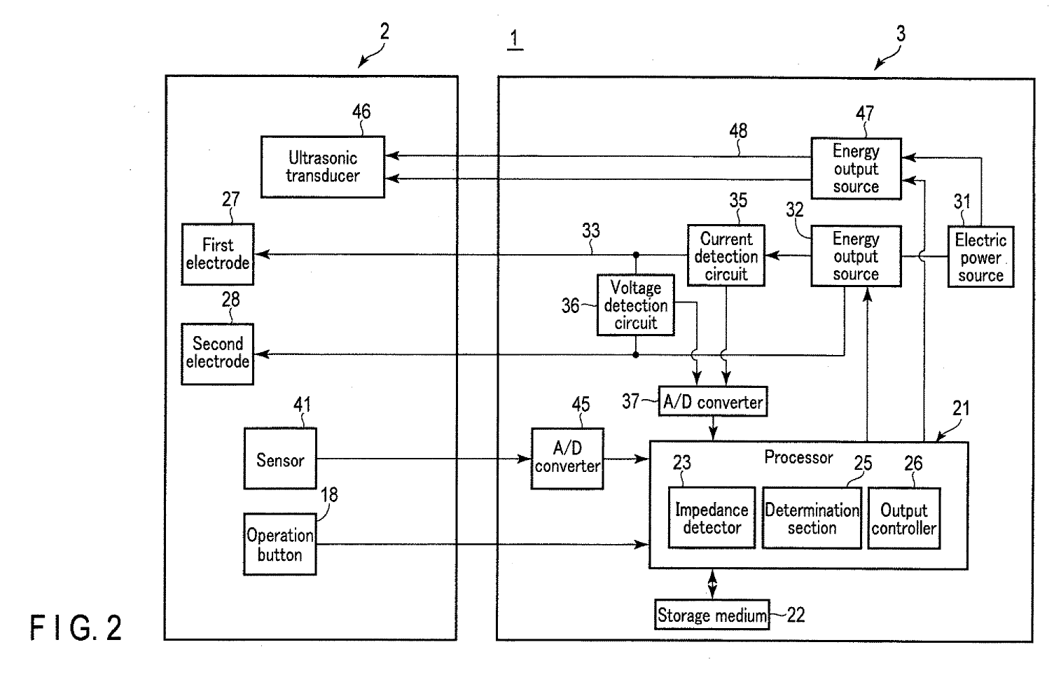

FIG. 2 is a diagram illustrating a control configuration in the treatment system 1. As illustrated in FIG. 2, the control device 3 includes a processor (controller) 21, which controls the entire treatment system 1, and a storage medium 22. The processor 21 is formed of an integrated circuit including a Central Processing Unit (CPU), an Application Specific Integrated Circuit (ASIC), or a Field Programmable Gate Array (FPGA). The processor 21 may be formed of a single integrated circuit, or of a plurality of integrated circuits. The process in the processor 21 is executed according to a program stored in the processor 21 or storage medium 22. The storage medium 22 stores a processing program for use in the processor 21, as well as parameters and tables for use in arithmetic process in the processor 21. The processor 21 includes an impedance detector 23, a determination section 25 and an output controller 26. The impedance detector 23, determination section 25 and output controller 26 function as parts of the processor 21, and execute some of the processes executed by the processor 21.

In the end effector 7 of the energy treatment instrument 2, the first grasping piece 15 is provided with a first electrode 27, and the second grasping piece 16 is provided with a second electrode 28. The electrodes 27 and 28 are formed of an electrically conductive material. The control device 3 includes an electric power source 31, which may be a battery or a power receptacle, and an energy output source (first energy output source) 32. The energy output source 32 is electrically connected to the electrodes 27 and 28 via an electricity supply path (first electricity supply path) 33 that extends inside the cable 10. The energy output source 32 includes a converter circuit, an amplifier circuit, and the like, and converts the electric power supplied from the electric power source 31. The energy output source 32 outputs the converted electric energy (high-frequency electric power). The electric energy that is output from the energy output source 32 is supplied to the electrodes 27 and 28 through the electricity supply path 33. The output controller 26 of the processor 21 controls the driving of the energy output source 32, and controls the output of the electric energy from the energy output source 32. In this manner, any one of output electric power P, output current I and output voltage V of the energy output source 32 is adjusted, and the supply of the electric energy to the electrodes 27 and 28 is controlled.

The electric energy is supplied from the energy output source 32 to the electrodes 27 and 28 with a treatment target being grasped between the grasping pieces 15 and 16. A high-frequency current thereby flows between the electrodes 27 and 28 through the treatment target that is being grasped in contact with the electrodes 27 and 28. That is, the high-frequency current is supplied as treatment energy to the treatment target. Due to the high-frequency current flowing through the treatment target, heat is caused in the treatment target, and this heat denatures the treatment target. The treatment target, such as a blood vessel, is sealed (coagulated) by using the high-frequency current. As described above, with the electric energy supplied from the energy output source 32 to the electrodes 27 and 28 of the energy treatment instrument 2, the treatment energy (high-frequency current) is applied to the treatment target grasped between the grasping pieces 15 and 16. According to the present embodiment, the grasping pieces 15 and 16 function as an energy application section (energy applier) that applies the high-frequency current as treatment energy to the grasped treatment target (blood vessel).

The electricity supply path 33 is provided with a current detection circuit 35 and a voltage detection circuit 36. When the electric energy is being output from the energy output source 32, the current detection circuit 35 detects the output current I, and the voltage detection circuit 36 detects the output voltage V. The energy control device 3 is provided with an A/D converter 37. To this A/D converter 37, an analog signal relating to the current I detected by the current detection circuit 35, and an analog signal relating to the voltage V detected by the voltage detection circuit 36 are transmitted. The A/D converter 37 converts the analog signal relating to the current I and the analog signal relating to the voltage V to digital signals, and transmits the converted digital signals to the processor 21.

When the electric energy is being output from the energy output source 32, the processor 21 acquires information relating to the output current I and the output voltage V of the energy output source 32. The impedance detector 23 of the processor 21 detects the impedance of the electricity supply path 33 including the grasped treatment target (blood vessel) and the electrodes 27 and 28, based on the output current I and the output voltage V. In this manner, an impedance Z between the paired grasping pieces 15 and 16 (i.e. the impedance of the grasped treatment target) is detected.

As illustrated in FIG. 1, an operation button 18 is attached to the housing 5 to function as an energy operation input section. By pressing the operation button 18, an operation (signal) for outputting the electric energy from the energy output source 32 to the energy treatment instrument 2 is input to the control device 3. In place of the operation button 18 or in addition to the operation button 18, a foot switch or the like may be provided separately from the energy treatment instrument 2, as the energy operation input section. As illustrated in FIG. 2, the processor 21 detects the presence or absence of input of an operation from the energy operation input section such as the operation button 18. Based on the input of the operation by the operation button 18, the output controller 26 of the processor 21 controls the output of the electric energy from the energy output source 32.

A treatment system 1 is provided with a sensor 41. The sensor 41 detects a parameter related to a load σ that acts toward the opening side on one of the grasping pieces 15 and 16 with respect to the open-close direction. The first grasping piece 15 opens toward the arrow Y2 side, whereas the second grasping piece 16 opens toward the arrow Y1 side.

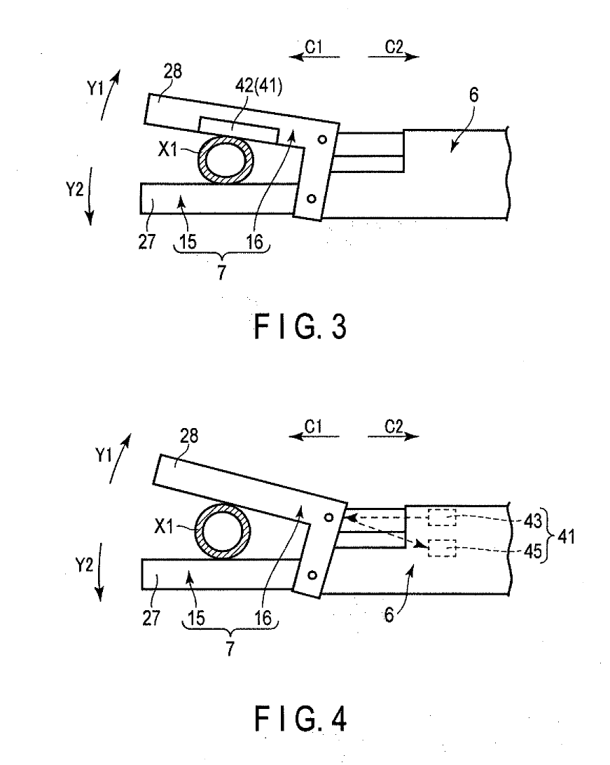

FIG. 3 illustrates an example of the sensor 41, and FIG. 4 illustrates another example of the sensor 41. In the example of FIG. 3, a pressure sensor 42 is provided as the sensor 41. In the state of a treatment target such as a blood vessel X1 being grasped between the grasping pieces and 16, the pressure sensor 42 detects the pressure acting on the pressure sensor 42 as a parameter related to a load σ acting toward the opening side on the second grasping piece 16. In the example of FIG. 4, the sensor 41 is provided with a light emitting element 43 and a light receiving element 44. In the state of the treatment target such as the blood vessel X1 being grasped between the grasping pieces 15 and 16, the light emitting element 43 emits laser light or the like toward the second grasping piece 16 (distal side) so that the light is reflected on the second grasping piece 16. The light receiving element receives the light reflected on the second grasping piece 16. Here, based on the intensity or the like of the light received by the light receiving element 44, the opening angle of the second grasping piece 16 with respect to the first grasping piece 15 is detected as a parameter related to the load σ that acts toward the opening side on the second grasping piece 16.

In the examples of FIGS. 3 and 4, a parameter of the load σ acting toward the opening side (arrow Y1 side) on the second grasping piece 16 is to be detected. A similar configuration can be adopted when a parameter of the load σ acting toward the opening side (arrow Y2 side) on the first grasping piece 15 is to be detected. Furthermore, in the examples of FIGS. 3 and 4, the sensor 41 is arranged in the energy treatment instrument 2, but the sensor 41 may be arranged separately from the energy treatment instrument 2.

As illustrated in FIG. 2, the energy control device 3 is provided with an A/D converter 45. An analog signal indicating a parameter related to a load σ detected by the sensor 41 is transmitted to the A/D converter 45. The A/D converter 45 converts the analog signal indicating the parameter related to the load σ to a digital signal, and transmits the converted digital signal to the processor 21. In one example, the A/D converter 45 may be arranged in the sensor 41. If this is the case, the analog signal indicating the parameter related to the load σ is converted to a digital signal by the sensor 41, and the converted digital signal is transmitted from the sensor 41 to the processor 21. Based on the detection result of the parameter related to the load σ in the sensor 41, the processor 21 calculates the load σ acting toward the opening side on one of the grasping pieces 15 and 16. For instance, when a pressure acting on the pressure sensor 42 of the second grasping piece 16 is to be detected as in the example of FIG. 3, the storage medium 22 may store therein a table or the like that represents the relationship between the pressure acting on the pressure sensor 42 and the load 6 acting toward the opening side on the second grasping piece 16. Based on the detection result of the pressure acting on the pressure sensor 42 and the table stored in the storage medium 22, the processor 21 calculates the load σ acting toward the opening side on the second grasping piece 16.

A determination section 25 of the processor 21 determines whether the load σ is smaller than a load threshold (threshold value) σth in one grasping piece (15 or 16) of the grasping pieces 15 and 16 for which the load σ is calculated. The load threshold σth may be set, for example, by the surgeon, or may be stored in the storage medium 22. Based on the detection result obtained by the sensor 41 and the determination result regarding the load σ, the output controller 26 of the processor 21 controls the output of the electric energy from the energy output source 32. In accordance with the output state of the electric energy from the energy output source 32, the actuation state of the energy treatment instrument 2 is switched between a first mode (first actuation mode) and a second mode (second actuation mode). According to the present embodiment, the state of the treatment energy (high-frequency current) applied from the energy application section (grasping pieces 15 and 16) to the grasped treatment target differs between the first mode and the second mode.

In one example, an ultrasonic transducer 46 may be provided in the energy treatment instrument 2 (inside the housing 5). If this is the case, a rod member is connected to the distal side of the ultrasonic transducer 46, and one of the grasping pieces 15 and 16 (e.g., the first grasping piece 15) is constituted by a projecting portion of this rod member that projects from the sheath 6 toward the distal side. In this example, in addition to the energy output source 32, an energy output source (second energy output source) 47 is provided in the control device 3. The energy output source 47 is electrically connected to the ultrasonic transducer 46 via an electricity supply path (second electricity supply path) 48 extending inside the cable 10. The energy output source 47 may be formed integrally with the energy output source 32, or may be formed separately from the energy output source 32.

In this example, the energy output source 47 includes a converter circuit, an amplifier circuit, and the like, and converts electric power from the electric power source 31. Then, the energy output source 47 outputs the converted electric energy (AC electric power). The electric energy that is output from the energy output source 47 is supplied to the ultrasonic transducer 46 through the electricity supply path 48. The output controller 26 of the processor 21 controls the driving of the energy output source 47, and controls the output of the electric energy from the energy output source 47.

In the present example, the electric energy (AC electric power) that is output from the energy output source 47 is supplied to the ultrasonic transducer 46 so that ultrasonic vibrations can be generated in the ultrasonic transducer 46. The generated ultrasonic vibrations are transmitted from the proximal side toward the distal side in the rod member (vibration transmitting member) so that the rod member including one of the grasping pieces 15 and 16 (e.g., first grasping piece 15) vibrates. By the rod member vibrating in the state of the treatment target being grasped between the grasping pieces 15 and 16, the ultrasonic vibrations are applied to the treatment target as treatment energy. At this time, frictional heat is generated from the vibrations, and the treatment target such as the blood vessel can be incised, while being sealed (coagulated), by use of the frictional heat.

In another example, a heater (not shown) may be provided, in place of the ultrasonic transducer 46, in the end effector 7 (at least one of the grasping pieces 15 and 16). If this is the case, the electric energy (DC electric power or AC electric power) that is output from the energy output source (47) is supplied to the heater through the electricity supply path (48). Heat is thereby generated by the heater, and the treatment target such as the blood vessel can be incised, while being sealed (coagulated), by use of the heat generated by the heater. When the ultrasonic vibration and the heat of the heater are applied as treatment energy to the grasped treatment target (blood vessel), at least one of the grasping pieces 15 and 16 still functions as the energy application section (energy applier) that applies the treatment energy to the treatment target.

Next, the function and advantageous effects of the present embodiment will be discussed. When a treatment is performed by using the treatment system 1, a surgeon holds the housing 5 of the energy treatment instrument 2, and inserts the end effector 7 into a body cavity such as an abdominal cavity. With the blood vessel (treatment target) being placed between the grasping pieces 15 and 16, the handle 12 is closed with respect to the grip 11 so that the grasping pieces 15 and 16 is closed relative to each other. In this manner, the blood vessel is grasped between the grasping pieces 15 and 16. With the blood vessel being grasped, the sensor 41 detects a parameter related to a load σ (for example, the pressure acting on the pressure sensor 42 (see FIG. 3)) that acts toward the opening side on one of the grasping pieces 15 and 16 (e.g. second grasping piece 16). Thereafter, a high-frequency current may be applied as treatment energy to the blood vessel so as to conduct a sealing treatment of the grasped blood vessel.

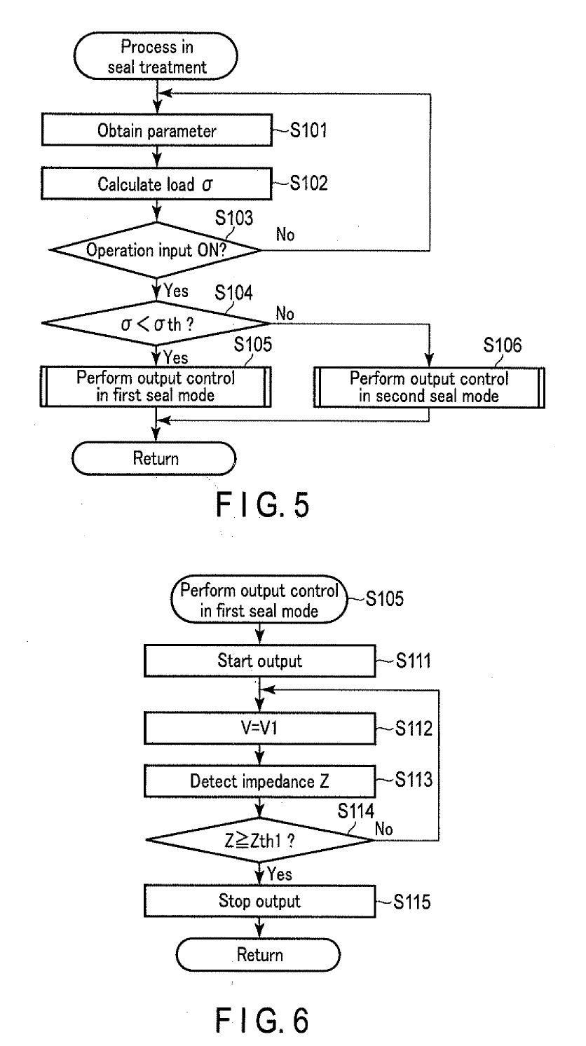

FIG. 5 is a flowchart illustrating a process executed in a seal treatment of a blood vessel by the processor 21 using the treatment system 1 of the present embodiment. As illustrated in FIG. 5, when performing the seal treatment of the blood vessel, the processor 21 obtains a parameter related to the load σ (for example, the pressure acting on the pressure sensor 42) that acts toward the opening side on one of the grasping pieces 15 and 16, with the blood vessel being grasped (step S101). In other words, the processor 21 obtains the detection result of the sensor 41 with the blood vessel being grasped between the grasping pieces 15 and 16. Based on the detection result of the obtained parameter, the processor 21 calculates the load o acting toward the opening side on one of the grasping pieces 15 and 16 (step S102). For instance, the storage medium 22 may store therein a table or the like that represents the relationship between the pressure acting on the pressure sensor 42 and the load σ acting toward the opening side on the second grasping piece 16, and the load σ is calculated using this table.

The processor 21 determines whether an operation input has been made using the operation button (energy operation input section) 18 (i.e., whether the operation input is ON or OFF) (step S103). If no operation input is made (No at step S103), the process returns to step 5101, where the processes of step 5101 and thereafter are sequentially executed. In this manner, the processes of obtaining the parameter related to the load σ and calculating the load σ are repeated. When the operation input is made (Yes at step S103), the determination section 25 of the processor 21 determines as to whether the calculated load 6 is smaller than the load threshold (threshold value) σth (step S104). That is, whether the load σ is greater than or equal to the load threshold σth is determined. Here, the determination is made based on the load σ at a time point when the operation input was switched from OFF to ON, or at a time point close to this time point. If the load σ is smaller than the load threshold σth (yes at step S104), the output controller 26 of the processor 21 executes the output control of the electric energy from the energy output source 32 in the first seal mode (step S105). If the load σ is greater than or equal to the load threshold σth (no at step S104), the output controller 26 executes the output control of the electric energy from the energy output source 32 in the second seal mode that is different from the first seal mode (step S106).

FIG. 6 is a flowchart indicating the process of the processor 21 in the output control in the first seal mode. As illustrated in FIG. 6, the processor 21 starts the output of the electric energy (high-frequency electric power) from the energy output source (first energy output source) 32 in the first seal mode of the output control (step S111). In this manner, the electric energy is supplied to the electrodes 27 and 28, and a high-frequency current flows to the grasped blood vessel, thereby sealing the blood vessel.

If a certain period of time has elapsed from the start of the output of the electric energy from the energy output source 32, the output controller 26 executes a constant voltage control to keep the output voltage V from the energy output source 32 constant at a first voltage V1 over time (step S112). Furthermore, when the output of the electric energy from the energy output source 32 is started, the impedance detector 23 of the processor 21 detects the impedance Z between the grasping pieces 15 and 16 (i.e. the impedance of the grasped treatment target), based on the detection result of the output current I obtained by the current detection circuit 35 and the detection result of the output voltage V obtained by the voltage detection circuit 36 (step S113). Then, the processor 21 determines whether a detected impedance Z is greater than or equal to an impedance threshold (first impedance threshold) Zth1 (step S114). The impedance threshold Zth1 may be set, for example, by the surgeon, or may be stored in the storage medium 22.

If the impedance Z is lower than the impedance threshold Zth1 (No at step S114), the process returns to step S112, and the processes of step S112 and thereafter are sequentially executed. If the impedance Z is greater than or equal to the impedance threshold Zth1 (Yes at step S114), the output controller 26 stops the output of the electric energy (high-frequency electric power) from the energy output source 32 (step S115). Thus, the supply of the electric energy to the electrodes 27 and 28 is stopped. The processor 21 executes the output control of the electric energy from the energy output source 32 in the first seal mode, and thereby the energy treatment instrument 2 is actuated in the first mode in which the grasped treatment target is coagulated (the blood vessel is sealed).

In the second seal mode of the output control, the processor 21 executes the processes of steps S111 and S113 to S115, similarly to the first seal mode of the output control. However, in the second seal mode, if a certain period of time has elapsed from the start of the output of the electric energy from the energy output source 32, the output controller 26 executes a constant voltage control for keeping the output voltage V from the energy output source 32 constant over time at a second voltage value V2, which is lower than the first voltage V1. Because the constant voltage control is executed at the second voltage value V2 that is lower than the first voltage V1, the electric energy that is output from the energy output source 32 is lower in the second seal mode than in the first seal mode. In other words, the output controller 26 of the processor 21 reduces the electric energy to be output from the energy output source 32 in the second seal mode, in comparison to the first seal mode. The processor 21 executes the output control of the electric energy from the energy output source 32 in the second seal mode so that the energy treatment instrument 2 is actuated in the second mode in which the grasped treatment target (seals the blood vessel) is coagulated and which is different from the first mode. As described above, in the present embodiment, the processor 21 controls the output of the electric energy from the energy output source 32, based on the determination result of the load σ, thereby switching the actuation state of the energy treatment instrument 2 between the first mode (first actuation mode) and the second mode (second actuation mode). The output state of the electric energy from the energy output source 32 differs between the first seal mode and the second seal mode. Thus, in the energy treatment instrument 2, the state of the treatment energy (high-frequency current) applied from the energy application section (grasping pieces 15 and 16) to the grasped treatment target differs between the first mode and the second mode.

As long as the electric energy to be output from the energy output source 32 becomes smaller in the second seal mode than in the first seal mode, the output control that is not the constant voltage control may be executed in the first seal mode and in the second seal mode. In one example, in the first seal mode, the output controller 26 may execute a constant electric power control to keep the output electric power P from the energy output source 32 constant over time at a first electric power P1. In the second seal mode, the output controller 26 executes a constant electric power control to keep the output electric power P from the energy output source 32 constant over time at a second electric power P2 that is lower than the first electric power P1. In another example, both the constant voltage control for keeping the output voltage V constant over time at the first voltage V1 and the constant electric power control for keeping the output electric power P constant over time at the first electric power P1 may be executed in the first seal mode, and switching is performed between the constant voltage control and the constant electric power control in accordance with the impedance Z. In the second seal mode, both the constant voltage control for keeping the output voltage V constant over time at the second voltage value V2 that is lower than the first voltage V1, and the constant electric power control for keeping the output electric power P constant over time at the second electric power P2 that is lower than the first electric power P1 may be executed, and switching is performed between the constant voltage control and the constant electric power control in accordance with the impedance Z. In any of the examples, the electric energy that is output from the energy output source 32 in the second seal mode is smaller than in the first seal mode.

According to the present embodiment, only the high-frequency current is applied as treatment energy to the blood vessel in each of the first seal mode and the second seal mode, and therefore the treatment energy other than the high-frequency current, such as ultrasonic vibrations and the heat of the heater, will not be applied to the blood vessel (treatment target). For instance, in the example in which the ultrasonic transducer 46 is provided in the energy treatment instrument 2, the processor 21 stops the output of the electric energy from the energy output source 47 to the ultrasonic transducer 46 in each of the first seal mode and the second seal mode. Thus, no electric energy is supplied to the ultrasonic transducer 46 in the first seal mode and second seal mode, and therefore no ultrasonic vibration will be generated by the ultrasonic transducer 46. Similarly, in the example in which a heater is provided in the energy treatment instrument 2, the processor 21 stops the output of the electric energy from the energy output source to the heater in each of the first seal mode and second seal mode. Thus, no electric energy is supplied to the heater in the first seal mode and second seal mode, and therefore no heat will be generated by the heater.

In one example, when the output control in the first seal mode or the output control in the second seal mode ends, no electric energy is supplied to the electrodes 27 and 28, the ultrasonic transducer 46, or the heater, and therefore no treatment energy, such as high-frequency current, ultrasonic vibrations, or the heat of the heater, will be applied to the treatment target. In another example, when the output control in the first seal mode or the output control in the second seal mode ends, the output control is automatically shifted to an incision mode. If this is the case, in the example in which the ultrasonic transducer 46 is provided in the energy treatment instrument 2, the processor 21 causes the energy output source 47 to output the electric energy to the ultrasonic transducer 46 at an incision level (high output level) in the incision mode. This causes the ultrasonic transducer 46 to produce ultrasonic vibrations and transmit the ultrasonic vibrations to one of the grasping pieces 15 and 16. The transmitted ultrasonic vibrations are applied as the treatment energy to the grasped blood vessel (treatment target), and the blood vessel is incised by frictional heat generated by the ultrasonic vibrations. Similarly, in the example in which the heater is provided in the energy treatment instrument 2, the processor 21 causes the energy output source to output the electric energy at the incision level (high output level) to the heater in the incision mode. The heater thereby generates heat. This heat of the heater is applied as the treatment energy to the grasped blood vessel, and the blood vessel is incised.

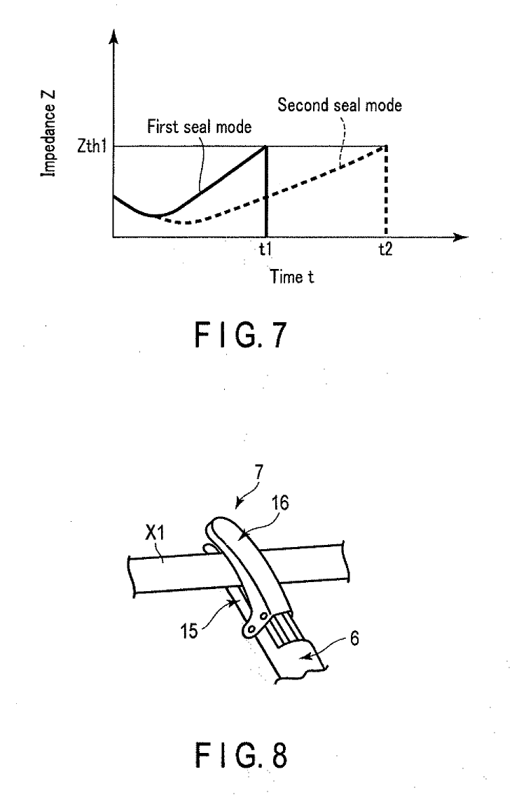

FIG. 7 is a diagram illustrating an example of a variation with time of the impedance Z between the paired grasping pieces 15 and 16 (i.e. the impedance of the grasped treatment target) in the state in which the processor 21 is executing the output control in the first seal mode and in the second seal mode. In FIG. 7, the ordinate axis indicates the impedance Z, and the abscissa axis indicates time t with reference to the start of the output of the electric energy from the energy output source 32. In FIG. 7, a solid line indicates a variation with time of the impedance Z in the first seal mode, and a broken line indicates a variation with time of the impedance Z in the second seal mode. As shown in FIG. 7, when the output of the electric energy from the energy output source 32 is started and the high-frequency current begins to flow to the blood vessel (treatment target), the impedance Z normally exhibits a behavior of decreasing with time for a certain length of time. After the impedance Z decreases over time to a certain level, the impedance Z normally exhibits a behavior of increasing over time in accordance with the rise in temperature of the treatment target due to the heat generated by the high-frequency current.

As described above, the electric energy that is output from the energy output source 32 in the second seal mode is lower than in the first seal mode according to the present embodiment. For this reason, in comparison with the first seal mode, the amount of heat generated per unit time due to the high-frequency current flowing in the blood vessel (treatment target) is smaller in the second seal mode. Accordingly, the rate of temperature rise of the treatment target (blood vessel) is lower, and the rate of increase of the impedance Z in the state in which the impedance Z increases with time is lower in the second seal mode than in the first seal mode. This means that the time length from the output start of the electric energy from the energy output source 32 to the time of the impedance Z reaching the impedance threshold Zth1 is longer in the second seal mode than in the first seal mode. In fact, in the example of FIG. 7, the impedance Z reaches the impedance threshold Zth1 at time t1 in the first seal mode, whereas the impedance Z reaches the impedance threshold Zth1 at time t2, which is later than time t1, in the second seal mode. As described above, in each of the first seal mode and second seal mode according to the present embodiment, the output of the electric energy from the energy output source 32 is stopped in accordance with the impedance Z reaching or exceeding the impedance threshold Zth1. For this reason, the output time length of the electric energy from the energy output source 32 is longer in the second seal mode than in the first seal mode.

As described above, in comparison to the first seal mode, the output controller 26 (processor 21) reduces the electric energy output from the energy output source 32, and increases the output time length of the electric energy from the energy output source 32 in the second seal mode. This means that, in comparison to the first seal mode, the amount of heat generated per unit of time due to the high-frequency current in the blood vessel is smaller, and the time length of the high-frequency current being supplied to the blood vessel is longer in the second seal mode. That is, in the energy treatment instrument 2, the time length of the treatment energy (high-frequency current) being applied from the energy application section (grasping pieces 15 and 16) to the treatment target (blood vessel) is longer in the second mode (second actuation mode) than in the first mode (first actuation mode). The total amount of treatment energy (high-frequency current) applied to the treatment target in the first seal mode corresponds to, for example, the area defined by the impedance Z indicated by the solid line and time t in FIG. 7. The total amount of treatment energy (high-frequency current) applied to the treatment target in the second seal mode corresponds to, for example, the area defined by the impedance Z indicated by the broken line in FIG. 7 and time t. In FIG. 7, the area on the lower side of the impedance Z in the second seal mode, defined by the broken line, is larger than the area on the lower side of the impedance Z in the first seal mode, defined by the solid line. The performance of sealing the blood vessel by the high-frequency current is therefore higher in the second seal mode than in the first seal mode.

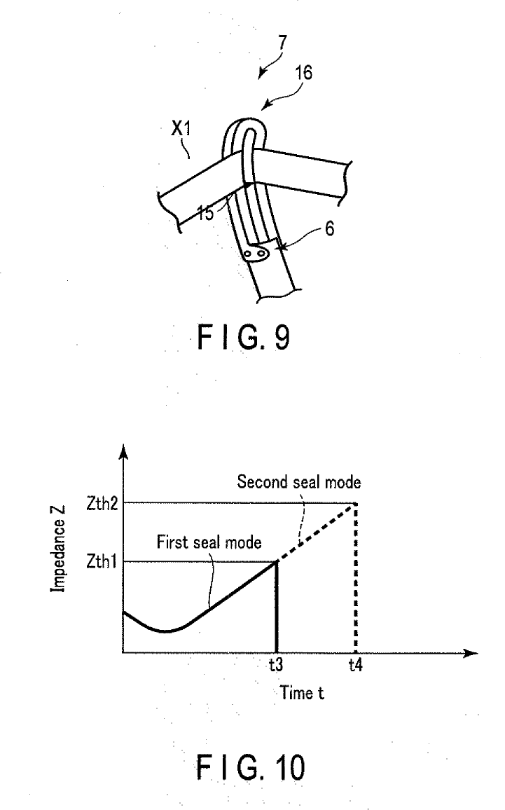

Each of FIGS. 8 and 9 is a diagram illustrating an example of a state in which a blood vessel X1 is grasped between the grasping pieces 15 and 16. When grasping the blood vessel X1, the blood vessel X1 may be grasped, as illustrated in FIG. 8, without being pulled in a direction intersecting (substantially perpendicular to) the extending direction of the blood vessel X1. Alternatively, as illustrated in FIG. 9, the blood vessel X1 may be grasped to be pulled to one side in the direction intersecting the extending direction of the blood vessel X1. In the state illustrated in FIG. 9, the blood vessel X1 is pulled to the side where the first grasping piece 15 is located according to the direction intersecting the extending direction of the blood vessel X1. As discussed above, when the blood vessel X1 is pulled, tension is exerted on the portion of the blood vessel X1 that is being pulled. For this reason, in the grasping piece (15 or 16) which is located on the side opposite to the side of the blood vessel X1 being pulled with respect to the blood vessel X1, the load σ to the opening side of the this grasping piece (15 or 16) is increased. In the state of FIG. 9, the load σ toward the opening side is increased in the second grasping piece 16 which is located on the side opposite to the side of the blood vessel X1 being pulled with respect to the blood vessel X1. When the load σ toward the opening side in one of the grasping pieces (15 or 16) increases, the treatment of sealing the grasped blood vessel X1 by using the treatment energy such as a high-frequency current may be affected. Thus, there is a possibility that the performance of sealing the blood vessel X1, as represented by a pressure resistance value of the sealed blood vessel X1, may be affected.

According to the present embodiment, the sensor 41 detects a parameter related to the load σ acting toward the opening side on one of the grasping pieces 15 and 16, and the processor 21 calculates the load G, based on the detection result obtained by the sensor 41. If the load o is smaller than the load threshold (threshold value) σth, the output control is executed in the first seal mode. If the load σ is greater than or equal to the load threshold σth, the output control is executed in the second seal mode.

Thus, in comparison to the case in which the load σ is smaller than the load threshold σth, the electric energy that is output from the energy output source 32 is smaller, and the output time length of the electric energy from the energy output source 32 is longer when the load σ is greater than or equal to the load threshold σth. That is, in the energy treatment instrument 2, the time length of the treatment energy (high-frequency current) supplied from the energy application section (grasping pieces 15 and 16) to the treatment target (blood vessel) is longer in the second mode (second actuation mode), when the load σ is greater than or equal to the load threshold σth, than in the first mode (first actuation mode), when the load σ is smaller than the load threshold σth. Thus, when the load o is greater than or equal to the load threshold σth, the treatment is performed in the second seal mode, in which the energy treatment instrument 2 of the treatment system 1 offers a sealing performance higher than in the first seal mode using the high-frequency current. Thus, the blood vessel can be sealed to the same degree as when the load σ is smaller than the load threshold σth. By using the energy treatment instrument 2 of the treatment system 1, the sealing performance of the blood vessel, as represented for example by a pressure resistance value of the sealed blood vessel (resistance of the blood flow to the sealed region), can be easily maintained even when the load σ is greater than or equal to the load threshold σth.

As described above, even when the load σ toward the opening side in one of the grasping pieces (15 or 16) increases, the grasped blood vessel can be suitably sealed by increasing the performance of sealing the blood vessel using the high-frequency current according to the present embodiment. That is, even when the blood vessel X1 is grasped while being pulled to one side in the direction intersecting the extending direction of the blood vessel X1, the blood vessel X1 can be suitably sealed using the treatment energy such as a high-frequency current, and a suitable treatment performance (sealing performance) can be achieved.

According to another exemplary embodiment, the process performed by the processor 21 in the second seal mode of the output control differs from the process in the previously described embodiment. For the first seal mode of the output control in this embodiment, the processor 21 executes the same process as the previously described embodiment (see FIG. 6). For the second seal mode of the output control also, the processor 21 executes the process of steps S111 to S113 in the same manner as in the first seal mode of the output control. In the second seal mode, however, the processor 21 determines whether the detected impedance Z is greater than or equal to an impedance threshold (second impedance threshold) Zth2, instead of executing the process of step S114. Here, the impedance threshold Zth2 is greater than the impedance threshold (first impedance threshold) Zth1. Further, the impedance threshold Zth2 may be set, for example, by the surgeon, or may be stored in the storage medium 22.

If the impedance Z is smaller than the impedance threshold Zth2, the process returns to step S112, where the processes of step S112 and thereafter are sequentially executed. If the impedance Z is greater than or equal to the impedance threshold Zth2, the output controller 26 stops the output of the electric energy (high-frequency electric power) from the energy output source 32. Accordingly, in the second seal mode of the present embodiment, the output of the electric energy from the energy output source 32 is stopped in response to the impedance Z having reached or exceeded the impedance threshold (second impedance threshold) Zth2, which is greater than the impedance threshold (first impedance threshold) Zth1. The processor 21 controls the output of the electric energy from the energy output source 32, based on the determination result of the load G, and thereby switches the actuation state of the energy treatment instrument 2 between the first mode (first actuation mode) and the second mode (second actuation mode) in this embodiment. Furthermore, in this embodiment also, the state of the electric energy output from the energy output source 32 is different between the first seal mode and the second seal mode. Thus, in the energy treatment instrument 2, the state of the treatment energy (high-frequency current) applied from the energy application section (grasping pieces 15 and 16) to the grasped treatment target differs between the first mode and the second mode.

FIG. 10 is a diagram illustrating an example of a variation with time of the impedance Z between the paired grasping pieces 15 and 16 in the state in which the processor 21 of this embodiment is executing the output control in the first seal mode and in the second seal mode. In FIG. 10, the ordinate axis indicates the impedance Z, and the abscissa axis indicates time t with reference to the start of the output of the electric energy from the energy output source 32. Furthermore, in FIG. 10, a solid line indicates a variation with time of the impedance Z in the first seal mode, and a broken line indicates a variation with time of the impedance Z in the second seal mode.

As described above, in the present embodiment, the output of the electric energy from the energy output source 32 is stopped in response to the impedance Z having reached or exceeded the impedance threshold Zth1 in the first seal mode. On the other hand, in the second seal mode, the output of the electric energy from the energy output source 32 is stopped in response to the impedance Z having reached or exceeded the impedance threshold Zth2. The impedance threshold Zth2 is greater than the impedance threshold Zth1. Thus, the output time length of the electric energy from the energy output source 32 is longer in the second seal mode than in the first seal mode. In fact, in the example of FIG. 10, the output of the electric energy is stopped at time t3 in the first seal mode, whereas the output of the electric energy is stopped at time t4, which is later than time t3 in the second seal mode.

As described above, in the present embodiment, the output controller 26 (processor 21) sets the impedance threshold (Zth2), which serves as the reference for stopping the output, to be larger and the output time length of the electric energy from the energy output source 32 to be longer, in the second seal mode than in the first seal mode. That is, in the energy treatment instrument 2 of the present embodiment, the time length of the treatment energy (high-frequency current) applied from the energy application section (grasping pieces 15 and 16) to the treatment target (blood vessel) is longer in the second mode (second actuation mode) in which the load σ is greater than or equal to the load threshold σth than in the first mode (first actuation mode) in which the load σ is smaller than the load threshold σth. Thus, in comparison to the first seal mode, the time length during which the high-frequency current is applied to the blood vessel is longer, and the total amount of treatment energy (high-frequency current) applied to the blood vessel is larger in the second seal mode, and the performance of sealing the blood vessel by the high-frequency current is thereby enhanced. Accordingly, in this embodiment, when the load σ is greater than or equal to the load threshold σth, the treatment is performed in the second seal mode, in which the performance of the energy treatment instrument 2 of the treatment system 1 sealing the blood vessel by use of the high-frequency current is higher than the first seal mode. Thus, the blood vessel can be sealed to substantially the same degree as when the load σ is smaller than the load threshold σth. By using the energy treatment instrument 2 of the treatment system 1, the blood vessel sealing performance as represented, for example, by a pressure resistance value of the sealed blood vessel (resistance to the blood flow to the sealed region) can be easily maintained even when the load σ is greater than or equal to the load threshold σth.

As one embodiment, the previously described embodiments may be combined. If this is the case, the processor 21 reduces the electric energy output from the energy output source 32, and sets the impedance threshold (Zth2), which serves as the reference for stopping the output, to be larger in the second seal mode, in comparison to the first seal mode. Since the state of the electric energy output from the energy output source 32 differs between the first seal mode and second seal mode in this embodiment, the state of the treatment energy (high-frequency current) applied from the energy application section (grasping pieces 15 and 16) to the grasped treatment target differs between the first mode and the second mode in the energy treatment instrument 2.

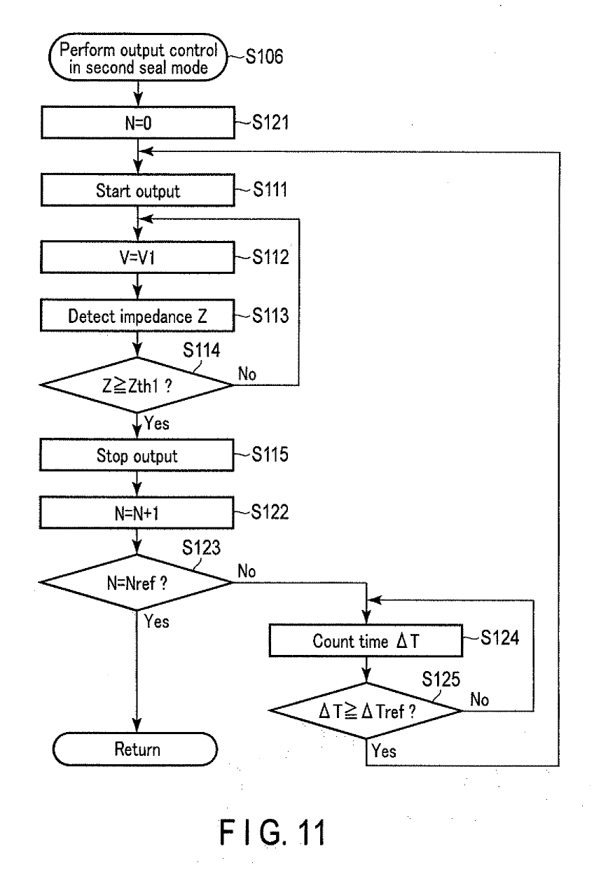

In another embodiment, the processor 21 executes a process illustrated in FIG. 11 in the second seal mode of the output control. In the first seal mode of the output control in the present embodiment, the processor 21 executes the same process as the embodiment shown in FIG. 6. In this embodiment, the number of outputs N is defined as a parameter for the electric energy from the energy output source 32 in the second seal mode of the output control. In the second seal mode of the output control, the processor 21 sets 0 as a default value for the number of outputs N (step S121). In the same manner as in the first seal mode of the output control, the processor 21 executes the processes of steps S111 to S115.

When the output of the electric energy from the energy output source 32 is stopped by the process at step S115, the processor 21 increments the number of outputs N by 1 (step S122). Then, the processor 21 determines whether the incremented number of outputs N is equal to a reference number of times Nref (step S123). The reference number of times Nref is any natural number greater than or equal to 2, which may be set, for example, by the surgeon, or may be stored in the storage medium 22. If the number of outputs N is equal to the reference number of times Nref, or in other words, if the number of outputs N has reached the reference number of times Nref (yes at step S123), the processor 21 terminates the output control in the second seal mode. In this manner, the state in which the output of the electric energy from the energy output source 32 is stopped can be continuously maintained.

Here, the time elapsed from the latest time point (time point 0) of the time points at which the output of the electric energy from the energy output source 32 is stopped by the process at step 5115 is defined as ΔT. If the number of outputs N is not equal to the reference number of times Nref, or in other words, if the output number of times N has not reached the reference number of times Nref (No at step S123), the processor 21 counts the time ΔT (step S124). Then, the processor 21 determines whether the counted time ΔT is greater than or equal to a reference time ΔTref (step S125). The reference time ΔTref may be, for example, 10 msec, which may be set, for example, by the surgeon, or may be stored in the storage medium 22.

If the time ΔT is shorter than the reference time ΔTref (No at step S125), the process returns to step 5124, and the processes of step 5124 and thereafter are sequentially executed. Specifically, the state in which the output of the electric energy from the energy output source 32 is stopped is maintained, and the time ΔT continues to be counted. If the time ΔT is the reference time ΔTref or greater (Yes at step S125), the process returns to step S111, and the processes of step S111 and thereafter are sequentially executed. In other words, the output of the electric energy from the energy output source 32 is resumed.

With the above process, in the second seal mode of the output control, the output controller 26 of the processor 21 stops the output of the electric energy after starting the output of the electric energy from the energy output source 32. Furthermore, after suspending the output of the electric energy from the energy output source 32, the output controller 26 resumes the output of the electric energy. That is, in the second seal mode, when the reference time ΔTref has passed after the time point of suspending the output of the electric energy from the energy output source 32, the electric energy is output once again from the energy output source 32. During the output control in the second seal mode, the processor 21 causes the energy output source 32 to intermittently output the electric energy for the reference number of times Nref (multiple times). The processor 21 controls, in the present embodiment, the output of the electric energy from the energy output source 32, based on the determination result of the load σ, and thereby switches the actuation state of the energy treatment instrument 2 between the first mode (first actuation mode) and the second mode (second actuation mode). The output state of the electric energy from the energy output source 32 differs between the first seal mode and second seal mode in this embodiment, and therefore the application state of the treatment energy (high-frequency current) from the energy application section (grasping pieces 15 and 16) to the grasped treatment target in the energy treatment instrument 2 differs between the first mode and the second mode.

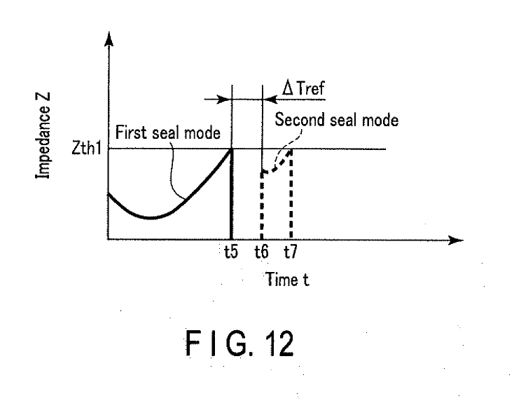

FIG. 12 is a diagram illustrating an example of a variation with time of the impedance Z between the paired grasping pieces 15 and 16 in the state in which the processor 21 of this embodiment is executing the output control in the first seal mode and in the second seal mode. In FIG. 12, the ordinate axis indicates the impedance Z, and the abscissa axis indicates time t with reference to the start of the output of the electric energy from the energy output source 32. Furthermore, in FIG. 12, a solid line indicates a variation with time of the impedance Z in the first seal mode, and a broken line indicates a variation with time of the impedance Z in the second seal mode. In the example shown in FIG. 12, the output of the electric energy from the energy output source 32 is stopped at time t5, in response to the impedance Z having reached the impedance threshold Zth1, in each of the first seal mode and second seal mode.

As described above, in the present embodiment, the electric energy is intermittently output from the energy output source 32 for multiple times (reference number of times Nref) in the second seal mode. In the second seal mode in the example shown in FIG. 12, the output of the electric energy from the energy output source 32 is resumed at time t6 when the reference time ΔTref has elapsed from time t5 at which the output was stopped. Here, the impedance Z is smaller than the impedance threshold Zth1. At time t7 after the time t6 (at which the output of the electric energy is resumed), in response to the impedance Z having reached the impedance threshold Zth1, the output of the electric energy from the energy output source 32 is stopped once again. In the example of FIG. 12, the reference number of times Nref is 2.

As described above, in the present embodiment, the output controller 26 (processor 21) resumes the output of the electric energy after suspending the output in the second seal mode. The output time length of the electric energy from the energy output source 32 therefore becomes longer in the second seal mode than in the first seal mode, as a result of which the time length of the high-frequency current being applied to the blood vessel becomes longer in the second seal mode than in the first seal mode. That is, in the energy treatment instrument 2 of the present embodiment, the time length of the treatment energy (high-frequency current) being applied from the energy application section (grasping pieces 15 and 16) to the treatment target (blood vessel) is longer in the second mode (second actuation mode) in which the load σ is greater than or equal to the load threshold σth than in the first mode (first actuation mode) in which the load σ is smaller than the load threshold σth. For this reason, the performance of sealing the blood vessel by the high-frequency current is higher in the second seal mode than in the first seal mode. Accordingly, when the load σ is greater than or equal to the load threshold σth, the treatment is performed in the second seal mode, in which the performance of sealing the blood vessel using the high-frequency current of the energy treatment instrument 2 of the treatment system 1 is higher than in the first seal mode. Thus, the blood vessel can be sealed to substantially the same degree as when the load σ is smaller than the load threshold σth. By using the energy treatment instrument 2 of the treatment system 1, the performance of sealing the blood vessel, as represented, for example, by the pressure resistance value of the sealed blood vessel (resistance of the blood flow to the sealed region), can be readily maintained even when the load σ is greater than or equal to the load threshold σth.

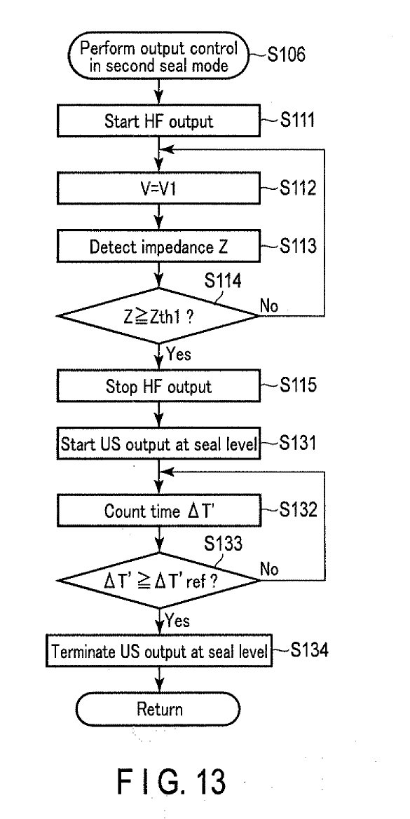

In another embodiment, the processor 21 executes a process as illustrated in FIG. 13 in the second seal mode of the output control. In the present embodiment, in the first seal mode of the output control, the processor 21 executes the same process as in the embodiment shown in FIG. 6. Furthermore, in the second seal mode of the output control, the processor 21 executes the processes of steps S111 through S115 in the same manner as in the first seal mode of the output control.

In the second seal mode, when the output of the electric energy from the energy output source 32 is stopped as a result of the process in step S115, the output controller 26 of the processor 21 starts the output of the electric energy from the energy output source 47 to the ultrasonic transducer 46 (step S131). Here, the energy output source 47 outputs the electric energy at a seal level having a low output level. That is, when the electric energy is output at the seal level, the output level is lower than the output of the electric energy at the above-described incision level. Thus, the electric energy supplied to the ultrasonic transducer 46 is lower, and the amplitude of the ultrasonic vibrations transferred to one of the grasping pieces 15 and 16 is smaller, in the output at the seal level than in the output at the incision level. Thus, the amount of frictional heat generated by the ultrasonic vibrations is small in the output at the seal level, and thereby the grasped blood vessel will not be incised by the frictional heat, but will only be sealed. In FIG. 13, the “HF output” denotes the high-frequency output of the electric energy from the energy output source 32 to the electrodes 27 and 28, and the “US output” denotes the ultrasonic output of the electric energy from the energy output source 47 to the ultrasonic transducer 46.

Here, a time (elapsed time) ΔT′ is defined with reference to the time point of starting the output of the electric energy from the energy output source 47 at the seal level as a result of the process in step S131 (i.e., the time point of stopping the output from the energy output source 32 as a result of the process in step S115) being 0. When the output of the electric energy is started from the energy output source 47 at the seal level, the processor 21 starts counting the time ΔT′ (step S132). The processor 21 determines whether the counted time ΔT′ is greater than or equal to a reference time ΔT′ref (step S133). The reference time ΔT′ref may be set, for example, by the surgeon, or may be stored in the storage medium 22.

If the time ΔT′ is shorter than the reference time ΔT′ref (No at step S133), the process returns to step S132, and the processes of step S132 and thereafter are sequentially executed. That is, the time ΔT′ continues to be counted. If the time ΔT′ is greater than or equal to the reference time ΔT′ref (Yes at step S133), the output controller 26 terminates the output of the electric energy from the energy output source 47 at the seal level (step S134). Here, the output of the electric energy from the energy output source 47 to the ultrasonic transducer 46 may be stopped. Alternatively, the output control may be automatically shifted to the incision mode so as to automatically change to a state in which the electric energy is output to the ultrasonic transducer 46 at the incision level (high output level). In one example, instead of the processes of step S132 and S133, the output controller 26 may terminate the output of the electric energy at the seal level from the energy output source 47, in response to the release of the operation input of the operation button (energy operation input section) 18 (i.e. the operation input being turned off).

As described above, in the present embodiment, when the output controller 26 (processor 21) stops the output of the electric energy to the electrodes 27 and 28 in the second seal mode, the output controller 26 starts the output of the electric energy to the ultrasonic transducer 46. That is, the processor 21 controls the output of the electric energy from the energy output sources 32 and 47, based on the determination result of the load σ, thereby switching the actuation state of the energy treatment instrument 2 between the first mode (first actuation mode) and the second mode (second actuation mode). In the present embodiment, the electric energy is output from the energy output source 47 only in the second seal mode. Thus, in the energy treatment instrument 2, the state of the treatment energy (high-frequency current and ultrasonic vibrations) applied from the energy application section (grasping pieces 15 and 16) to the grasped treatment target differs between the first mode and the second mode. For this reason, in the second seal mode, even after the output of the electric energy to the electrodes 27 and 28 is stopped, the ultrasonic vibrations (frictional heat) seal the grasped blood vessel. That is, in the second seal mode, even in the state in which the impedance Z is increased, causing a resistance to the high-frequency current flow in the blood vessel, the blood vessel can still be sealed by the frictional heat generated by the ultrasonic vibrations. In this manner, in comparison to the first seal mode, the performance of sealing the blood vessel by the treatment energy is enhanced in the second seal mode. Accordingly, when the load σ is greater than or equal to the load threshold σth, the treatment is performed in the second seal mode, in which the performance of sealing the blood vessel using the treatment energy of the energy treatment instrument 2 of the treatment system 1 is higher than in the first seal mode. Thus, the blood vessel can be sealed to substantially the same degree as when the load σ is smaller than the load threshold σth. By using the energy treatment instrument 2 of the treatment system 1, the performance of sealing the blood vessel as represented, for example, by the pressure resistance value of the sealed blood vessel (resistance of the blood flow to the sealed region), can be readily maintained even when the load σ is greater than or equal to the load threshold σth.

In one embodiment, when the output of the electric energy from the energy output source 32 is stopped by the process in step S115 in the second seal mode, the output controller 26 of the processor 21 starts the output of the electric energy to the heater. At this time, the electric energy is output at the seal level having a lower output level than the above-described incision level. Thus, the electric energy supplied to the heater as the output at the seal level is smaller than the output at the incision level. With a small amount of heat generated by the heater as the output at the seal level, the grasped blood vessel is not incised by the heat of the heater, and therefore only sealing of the blood vessel is performed. In this embodiment, the blood vessel is sealed in the second seal mode by the heat of the heater in addition to the high-frequency current. That is, in the present embodiment, the state of the treatment energy (the high-frequency current and the heat of the heater) applied from the energy application section (grasping pieces 15 and 16) to the grasped treatment target differs between the first mode and the second mode in the energy treatment instrument 2. The performance of sealing the blood vessel by the treatment energy is therefore higher in the second seal mode than in the first seal mode. Thus, the same function and advantageous effects as in the embodiment shown in FIG. 13 can be obtained.

The output control of the electric energy, in which the sealing performance of the blood vessel by the treatment energy is increased when the load σ is greater than or equal to the load threshold σth in comparison to when the load σ is smaller than the load threshold σth, may be adopted for an example in which a high-frequency current is not applied to the blood vessel, and only the treatment energy other than the high-frequency current (e.g., the ultrasonic vibration and the heat of the heater) is applied to the blood vessel. For instance, in one embodiment in which the electric energy is output to the ultrasonic transducer 46 at the seal level so as to seal the blood vessel by using only the ultrasonic vibrations, the processor 21 reduces the electric energy to be output from the energy output source 47 to the ultrasonic transducer 46, and increases the output time length of the electric energy to the ultrasonic transducer 46 in the second seal mode (the second mode of the energy treatment instrument 2), in comparison to the first seal mode (the first mode of the energy treatment instrument 2). In this manner, the time length of the ultrasonic vibrations being applied to the blood vessel is longer, and the performance of sealing the blood vessel by the ultrasonic vibrations is higher in the second seal mode (when the load σ is greater than or equal to the load threshold σth) than in the first seal mode (when the load σ is smaller than the load threshold σth). Furthermore, in one embodiment in which the electric energy is output to the heater at the seal level and the blood vessel is sealed by using only the heat of the heater, the processor 21 reduces the electric energy to be output from the energy output source to the heater, and increases the output time length of the electric energy to the heater in the second seal mode, in comparison with the first seal mode. As a result, the time length of the heat of the heater being applied to the blood vessel becomes longer, and the performance of sealing the blood vessel by the heat of the heater becomes higher in the second seal mode (when the load σ is greater than or equal to the load threshold σth) than in the first seal mode (when the load σ is smaller than the load threshold σth). With the energy treatment instrument 2 of the treatment system 1, the performance of sealing the blood vessel as represented, for example, by the pressure resistance value of the sealed blood vessel (resistance to the blood flow to the sealed region) can be easily maintained even when the load σ is greater than or equal to the load threshold σth.

In one embodiment, whether the processor 21 executes the output control in the first seal mode or in the second seal mode may be determined, for example, by the surgeon. In this embodiment, for example, two operation buttons, which serve as an energy operation input section, may be provided so that, when an operation is input from one of the operation buttons, the processor 21 (output controller 26) executes the output control of the electric energy in the first seal mode, and the energy treatment instrument 2 is actuated in the first mode (first actuation mode) for coagulating the treatment target. When an operation is input from the other operation button, the processor 21 executes the output control of the electric energy in the second seal mode, in which the performance of sealing the blood vessel by the treatment energy is higher than in the first seal mode. The energy treatment instrument 2 is thereby actuated in the second mode (second actuation mode) in which the treatment target is coagulated and in which the state of the treatment energy applied to the treatment target differs from the first mode. In the second mode, the performance of coagulating the treatment target by the treatment energy (the performance of sealing the blood vessel by the treatment energy) is higher than in the first mode. In this embodiment, a notification section (not shown) may be provided in the control device 3 configured to notify whether the load σ acting on one of the grasping pieces 15 and 16 is smaller than the load threshold σth. In one example, the notification section is an LED, and the LED is turned on when the load σ is greater than or equal to the load threshold σth. In another example, the notification section may be a buzzer, a display screen, or the like.

In another embodiment, the notification section may be a display screen or the like configured to notify the detection result of a parameter related the load σ obtained by the sensor 41, or the load σ calculated by the processor 21. In this embodiment, the surgeon determines whether or not the load σ is smaller than the load threshold σth, based on the information notified by the notification section. Then, the surgeon determines which of the two operation buttons is to be operated to execute the operation input, and selects whether the processor 21 executes the output control in the first seal mode or in the second seal mode.

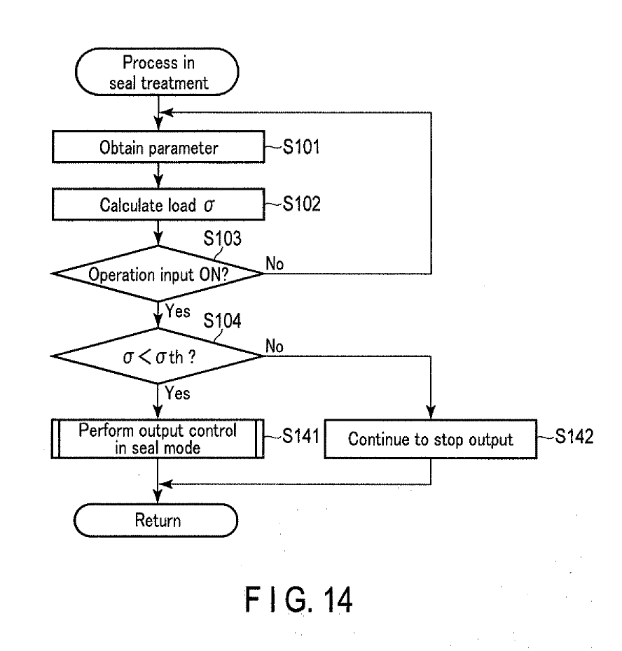

In another embodiment, in the seal treatment of the blood vessel, the processor 21 executes a process illustrated in FIG. 14. In the same manner as the above-described embodiment, the processor 21 executes the processes of steps 5101 to 5104 in the seal treatment of the blood vessel, in the present embodiment. When the load σ is smaller than the load threshold σth (Yes at step S104), the processor 21 executes the output control of the electric energy in the seal mode (step S141). In the output control in the seal mode, the processor 21 executes, for example, the same process as the output control in the first seal mode of the embodiment shown in FIG. 6. The processor 21 executes the output control of the electric energy in the seal mode, and thereby the energy treatment instrument 2 is actuated in the first mode for coagulating the grasped treatment target (sealing the blood vessel). If the load σ is greater than or equal to the load threshold σth (no at step S104), the processor 21 continues to stop the output of the electric energy, whether or not an operation is input from the operation button 18 (step S142). Here, the energy treatment instrument 2 is actuated in the second mode. That is, the output of the electric energy from the energy output sources 32 and 47 continues to be stopped. Thus, when the load σ is greater than or equal to the load threshold σth, no treatment energy such as high-frequency current is applied to the grasped blood vessel even if an operation is input from the operation button 18. Thus, in this embodiment, the processor 21 controls the output of the electric energy from the energy output source 32 based on the determination result of the tension, and thereby switches the actuation state of the energy treatment instrument 2 between the first mode (first actuation mode) and the second mode (second actuation mode). In this embodiment, the output of the electric energy from the energy output sources 32 and 47 is stopped in the second mode. Thus, the state of the treatment energy (high-frequency current, etc.) applied from the energy application section (grasping pieces 15 and 16) to the grasped treatment target in the energy treatment instrument 2 differs between the first mode and the second mode.

With the output control as described above in the present embodiment, no treatment energy is applied to the blood vessel when a large load σ is applied toward the opening side on one of the grasping pieces (15 or 16). In other words, in the state in which the sealing performance may be affected, for example, in the state in which the blood vessel is being pulled to one side in a direction intersecting the extending direction of the blood vessel, no treatment energy is applied to the blood vessel. The treatment energy is applied to the blood vessel only in the state in which the sealing performance will be barely affected, for example when the blood vessel is not being pulled. Thus, the blood vessel is suitably sealed by using the treatment energy such as a high-frequency current, and a suitable treatment performance (sealing performance) is achieved.

In still another embodiment, the surgeon may decide whether or not the electric energy should be output in the seal mode. In this embodiment, the above-described notification section may be provided in, for example, the control device 3. When it is notified or determined that the load σ is smaller than the load threshold σth, the surgeon inputs an operation from the operation button 18 so that the processor 21 executes the output control in the seal mode. The electric energy is thereby output from the energy output sources 32 and 47, and the energy treatment instrument 2 is actuated in the first mode (first actuation mode). On the other hand, when it is notified or determined that the load σ is greater than or equal to the load threshold σth, the surgeon will not input an operation from the operation button 18. Thus, without any electric energy output from the energy output sources 32 and 47, the energy treatment instrument 2 is actuated in the second mode (second actuation mode) that is different from the first mode.

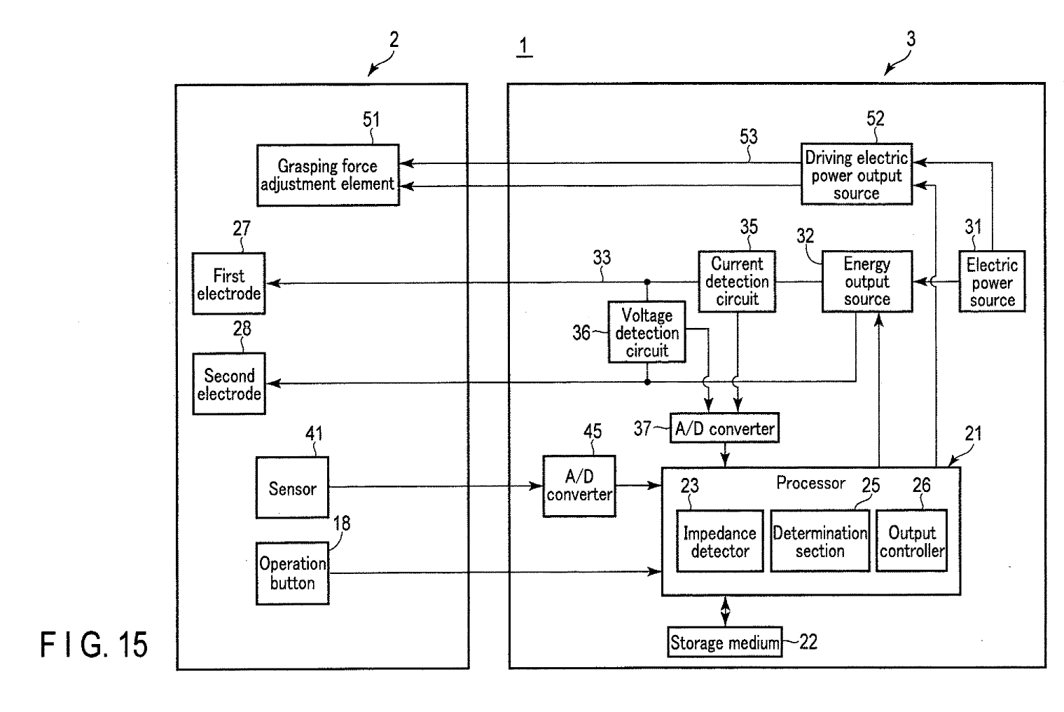

Next, another exemplary embodiment will be described with reference to FIGS. 15 to 17.

FIG. 15 is a diagram illustrating a control configuration in a treatment system 1 according to the present embodiment. In the present embodiment, a grasping force adjustment element (grasping force adjuster) 51 is provided in the energy treatment instrument 2, as illustrated in FIG. 15. A grasping force acting on the treatment target (blood vessel) between the grasping pieces 15 and 16 varies in accordance with a driving state of the grasping force adjustment element 51. That is, the grasping force acting on the treatment target between the grasping pieces 15 and 16 is adjusted by the grasping force adjustment element 51. In addition, in this embodiment, a driving electric power output source 52 is provided in the control device 3. The driving electric power output source is electrically connected to the grasping force adjustment element 51 via an electricity supply path 53 extending inside the cable 10. Here, the driving electric power output source 52 may be formed integrally with the above-described energy output sources 32 and 47, or may be formed separately from the energy output sources 32 and 47.

The driving electric power output source 52 includes a converter circuit, an amplifier circuit, and the like, and converts the electric power from the electric power source to the driving electric power for the grasping force adjustment element 51. The driving electric power output source 52 outputs the converted driving electric power, and the output driving electric power is supplied to the grasping force adjustment element 51 through the electricity supply path 53. The processor 21 controls the driving of the driving electric power output source 52, and controls the output of the driving electric power from the driving electric power output source 52. In this manner, the supply of the driving electric power to the grasping force adjustment element 51 is controlled, and the driving of the grasping force adjustment element 51 is controlled. According to the present embodiment, in accordance with the driving state of the grasping force adjustment element 51, the actuation state of the energy treatment instrument 2 is switched between the first mode (first actuation mode) and the second mode (second actuation mode). According to the present embodiment, the grasping force acting on the treatment target (blood vessel) between the grasping pieces 15 and 16 differs between the first mode and the second mode.

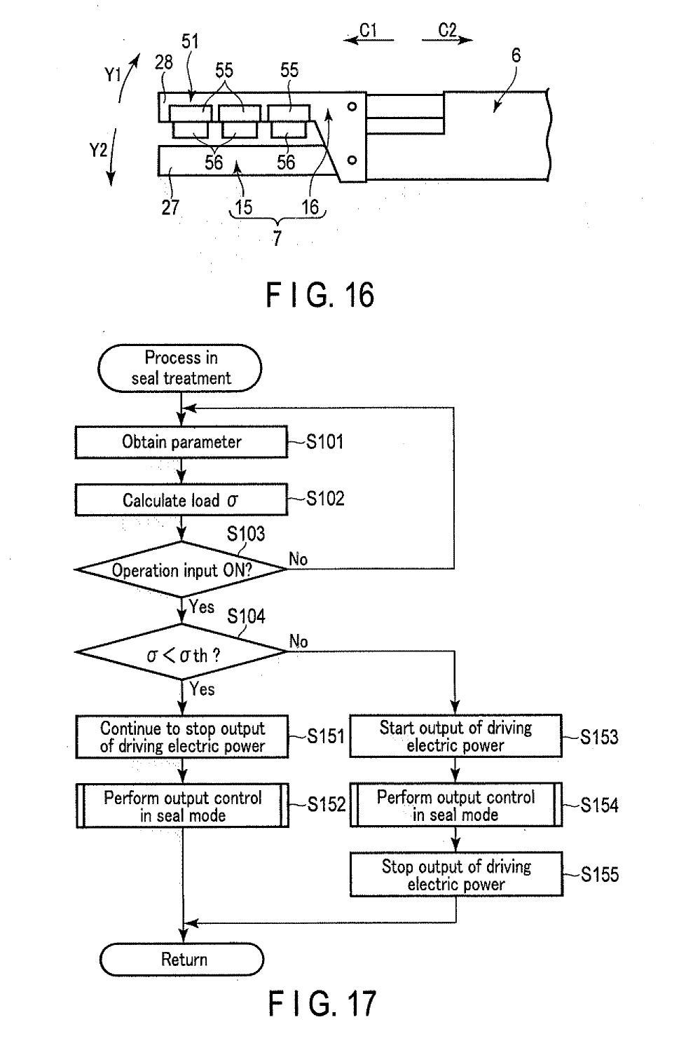

FIG. 16 is a diagram illustrating an example of the grasping force adjustment element 51. In the example illustrated in FIG. 16, a heater 55 and a volume change portion 56 are provided as the grasping force adjustment element 51 in the second grasping piece 16. The volume change portion 56 is formed of an electrically insulating material such as parylene, nylon, or ceramics. By closing the grasping pieces 15 and 16 relative to each other, the volume change portion 56 is brought into contact with the first grasping piece 15 (first electrode 27). In the state in which the volume change portion 56 is in contact with the first grasping piece 15, the electrodes 27 and 28 are spaced apart from each other, and are prevented from being in a contact with each other by the volume change portion 56. The volume change portion 56 is formed of a material having a high thermal expansion coefficient.

With the driving electric power output from the driving electric power output source 52 to the heater 55, the grasping force adjustment element 51 is driven, and heat is generated by the heater 55. With the heat generated by the heater 55, the temperature of the volume change portion 56 rises, as a result of which the volume change portion 56 expands (the volume of the volume change portion increases). Because of the volume change portion 56 expanding in the state in which the blood vessel (treatment target) is grasped between the grasping pieces 15 and 16, the distance between the grasping pieces 15 and 16 decreases, and the grasping force acting on the treatment target between the grasping pieces 15 and 16 increases. In this example, the heat generated by the heater 55 is not used for coagulation or incision of the treatment target.

In another example, a Peltier element may be provided in place of the heater 55. With this arrangement, the driving electric power is output from the driving electric power output source 52 to the Peltier element, and the Peltier element thereby transfers the heat to the volume change portion 56 side. With the heat transferred by the Peltier element, the temperature of the volume change portion 56 rises, as a result of which the volume change portion 56 expands. Thus, as described above, in the state in which the blood vessel (treatment target) is grasped between the grasping pieces 15 and 16, the distance between the grasping pieces 15 and 16 decreases, and the grasping force of the treatment target between the grasping pieces 15 and 16 increases.

Next, the function and advantageous effects of the present embodiment will be described. FIG. 17 is a flowchart illustrating the process executed by the processor 21 in the seal treatment of the blood vessel using the treatment system 1 of the present embodiment. In the present embodiment, the processor 21 executes the processes of steps S101 to S104 in the seal treatment of the blood vessel in the same manner as the above-described embodiment and the like. When the load σ is smaller than the load threshold σth (yes at step S104), the processor 21 continues to stop the output of the driving electric power from the driving electric power output source 52 to the grasping force adjustment element 51 (step S151). The grasping force adjustment element 51 is therefore not driven, and the volume change portion 56 does not expand. The grasping force of the treatment target between the grasping pieces 15 and 16 is thereby maintained. Furthermore, the processor 21 executes the output control of the electric energy from the energy output source 32 or the like in the seal mode (step S152). In the output control in the seal mode, the processor 21 executes, for example, the same process as the output control in the first seal mode of the embodiment shown in FIG. 6. In the state in which the output of the driving electric power from the driving electric power output source 52 to the grasping force adjustment element 51 is stopped by the processor 21 and the grasping force adjustment element 51 is not driven, the energy treatment instrument 2 is actuated in the first mode (first actuation mode) for coagulating the grasped treatment target (sealing the blood vessel).

On the other hand, when the load σ is greater than or equal to the load threshold σth (no at step S104), the processor 21 starts to output the driving electric power from the driving electric power output source 52 to the grasping force adjustment element 51 (step S153). Thus, the grasping force adjustment element 51 is driven, and the volume change portion 56 expands. The grasping force acting on the treatment target between the grasping pieces 15 and 16 thereby increases. The processor 21 executes the output control of the electric energy from the energy output source 32 or the like in the seal mode (step S154). In the output control in the seal mode, the processor 21 executes, for example, the same process as the output control in the first seal mode of the embodiment shown in FIG. 6. When the output control in the seal mode ends, the processor 21 stops the output of the driving electric power from the driving electric power output source 52 to the grasping force adjustment element 51 (step S155). In the state in which the processor 21 causes the driving electric power output source 52 to output the driving electric power to the grasping force adjustment element 51 and thereby drives the grasping force adjustment element 51, the energy treatment instrument 2 is actuated in the second mode (second actuation mode), which is different from the first mode and which is for coagulating the grasped treatment target (sealing the blood vessel). As described above, in the present embodiment, the processor 21 controls the output of the driving electric power from the driving electric power output source 52 based on the determination result of the load σ, thereby switching the actuation state of the energy treatment instrument 2 between the first mode (first actuation mode) and the second mode (second actuation mode). In the energy treatment instrument 2, the driving state of the grasping force adjustment element 51 differs between the first mode and the second mode. Thus, the grasping force of the treatment target (blood vessel) between the grasping pieces 15 and 16 differs between the first mode and the second mode.

In the present embodiment, under the control by the processor 21 as described above, the processor 21 increases the grasping force of the blood vessel (treatment target) between the grasping pieces 15 and 16 when the load σ is greater than or equal to the load threshold σth, in comparison to when the load σ is smaller than the load threshold σth. That is, in the energy treatment instrument 2, the grasping force acting on the blood vessel (treatment target) between the grasping pieces 15 and 16 is larger in the second mode (second actuation mode) than in the first mode (first actuation mode). For this reason, even when the load σ toward the opening side on one of the grasping pieces (15 or 16) is increased, the grasped blood vessel can be suitably sealed by increasing the grasping force acting on the blood vessel between the grasping pieces 15 and 16. That is, even when the blood vessel is grasped while being pulled to one side in the direction intersecting the extending direction of the blood vessel, the blood vessel can be suitably sealed using the treatment energy, and a suitable treatment performance (sealing performance) can be achieved.

The grasping force adjustment element 51 is not limited to the above configuration. In an exemplary embodiment, for example, an electric motor and an abutment member are provided as the grasping force adjustment element 51. If this is the case, the handle 12 is brought into contact with the abutment member by closing the handle 12 relative to the grip 11, and the handle 12 is closed relative to the grip 11 up to come to a position at which the handle 12 abuts on the abutment member. The processor (output controller 26) controls the output of the driving electric power from the driving electric power output source 52 to the electric motor, and thereby controls the driving of the electric motor. When the electric motor is driven, the abutment member is moved, and the position of the abutment member is changed. This changes the stroke of the handle at a time of closing the handle 12 relative to the grip 11. In the present embodiment, the processor 21 adjusts the position of the abutment member, based on a load σ so that the stroke of the handle 12 for closing is increased when the load σ is greater than or equal to the load threshold σth, in comparison to the case in which the load σ is smaller than the load threshold σth. Furthermore, in this embodiment, the grasping force for grasping the treatment target between the grasping pieces 15 and 16 increases when the load σ is greater than or equal to the load threshold σth (the second mode of the energy treatment instrument 2), in comparison to when the load σ is smaller than the load threshold σth (the first mode of the energy treatment instrument 2).

For an arrangement in which one of the grasping pieces 15 and 16 is formed by a rod member to be inserted through the sheath 6, a support member supporting the rod member on the most distal side within the sheath 6, and an electric motor or the like driven to move this support member, may be provided as the grasping force adjustment element 51. If this is the case, by driving the electric motor or the like in accordance with the load G, the position where the rod member is supported by the support member can be changed. In this manner, with the treatment target (blood vessel) being grasped between the grasping pieces 15 and 16, the amount of deflecting of the distal portion (one of the grasping pieces 15 and 16) of the rod member varies, and the grasping force between the grasping pieces 15 and 16 varies. In addition, the control for adjusting the grasping force may be suitably adopted, as long as the grasping force adjustment element 51 is provided for varying the grasping force acting on the treatment target (blood vessel) between the grasping pieces 15 and 16.

In another embodiment, an operation button or the like may be provided as a driving operation input section to output the driving electric power from the driving electric power output source 52. In this embodiment, the surgeon may decide as to whether or not the driving electric power should be output. In this embodiment, the above-described notification section may be provided, for example, in the control device 3. When it is notified or determined that the load σ is smaller than the load threshold σth, the surgeon will not input an operation from the operation button (driving operation input section). The driving electric power is therefore not output from the driving electric power output source 52 to the grasping force adjustment element 51 (heater 55), and the volume change portion 56 does not expand. Thus, the energy treatment instrument 2 is actuated in the first mode (first actuation mode). On the other hand, when it is notified or determined that the load σ is greater than or equal to the load threshold σth, the surgeon will input an operation from the operation button 18. In response, the driving electric power is output from the driving electric power output source 52 to the grasping force adjustment element 51 (heater 55), and the volume change portion 56 expands by the heat generated by the heater 55. Thus, the energy treatment instrument 2 is actuated in the second mode (second actuation mode), and the grasping force acting on the treatment target between the grasping pieces 15 and 16 increases.

In another exemplary embodiment, any of the disclosed embodiments may be combined. If this is the case, when the load σ is smaller than the load threshold σth, the processor 21 executes the output control of the electric energy from the energy output sources 32 and 47 in the first seal mode, and applies the treatment energy to the blood vessel. When the load σ is greater than or equal to the load threshold σth, the processor 21 executes the output control of the electric energy from the energy output sources 32 and 47 in the second seal mode, in which the performance of sealing the blood vessel by the treatment energy is higher than in the first seal mode, and the processor 21 applies the treatment energy to the blood vessel. That is, in this embodiment, the performance of sealing the blood vessel by the treatment energy is higher in the second mode of the energy treatment instrument 2 than in the first mode, in a manner similar to the embodiment shown in FIG. 6. Furthermore, in this embodiment, the processor 21 increases the grasping force acting on the treatment target between the grasping pieces 15 and 16 when the load σ is greater than or equal to the load threshold σth (the second mode of the energy treatment instrument 2), in comparison to when the load σ is smaller than the load threshold σth (the first mode of the energy treatment instrument 2).

In the above-described embodiments, an energy treatment instrument (2) of a treatment system (1) includes a first grasping piece (15), and a second grasping piece (16) configured to open and close relative to the first grasping piece (15) and configured to grasp a treatment target between the first grasping piece (15) and the second grasping piece (16). The actuation state of the energy treatment instrument (2) is switched between a first mode for coagulating a treatment target and a second mode for coagulating the treatment target that is different from the first mode, in accordance with a load (a) acting toward the opening side on one of the first grasping piece (15) and the second grasping piece (16). In the treatment system (1), the sensor (41) detects a parameter related the load (a) that acts toward the opening side on one of the first grasping piece (15) and the second grasping piece (16). An energy output source (32 or 47, or both 32 and 47) is configured to output the electric energy that is to be supplied to the energy treatment instrument (2), and is configured to apply the treatment energy to the treatment target grasped between the first grasping piece (15) and the second grasping piece (16) when the electric energy is supplied to the energy treatment instrument (2). The processor (21) determines, based on the detection result obtained by the sensor (41), as to whether the load (a) that acts toward the opening side on one of the first grasping piece (15) and the second grasping piece (16) is smaller than a threshold (σth). The processor (21) is configured to execute at least one of controlling an output of the electric energy from the energy output source (32 or 47, or both 32 and 47), based on the determination result regarding the load (σ), and increasing the grasping force acting on the treatment target between the first grasping piece (15) and the second grasping piece (16) when the load (σ) is greater than or equal to the threshold (σth), in comparison to when the load (σ) is smaller than the threshold (σth).

A characteristic feature is added below.

### (Addendum 1)

A treatment method comprising:

closing a first grasping piece and a second grasping piece with respect to each other, and grasping a treatment target between the first grasping piece and the second grasping piece;

obtaining a parameter related to a load acting toward an opening side on one of the first grasping piece and the second grasping piece, with the treatment target being grasped between the first grasping piece and the second grasping piece;

supplying electric energy from an energy output source to an energy treatment instrument, and applying treatment energy to the treatment target grasped between the first grasping piece and the second grasping piece;

determining, based on the obtained parameter, whether the load acting toward the opening side on the one of the first grasping piece and the second grasping piece is smaller than a threshold; and

performing at least one of controlling, based on a determination result of the load, the output of the electric energy from the energy output source, and increasing a grasping force for grasping the treatment target between the first grasping piece and the second grasping piece when the load is greater than or equal to the threshold, in comparison to when the load is smaller than the threshold.

Additional advantages and modifications will readily occur to those skilled in the art. Therefore, the broader aspects of the treatment system, control device, and treatment method are not limited to the specific details and embodiments shown and described herein. Accordingly, various modifications may be made without departing from the spirit or scope of the general inventive concept as defined by the appended claims and their equivalents.

1. A treatment system, comprising:
a treatment instrument including:
a first grasping piece that includes a first electrode;
a second grasping that includes a second electrode that is different from the first electrode, the second grasping piece being configured to grasp a treatment target together with the first grasping piece by opening and closing with respect to the first grasping piece; and
a sensor, and the sensor being configured to detect a load that acts on the second grasping piece; and
a control device configured to:
output electric energy to the first electrode and the second electrode:,
supply a high-frequency current to the treatment target for treating the treatment target;
switch between a first mode and a second mode that is different from the first mode in a control scheme such that outputs of the electric energy to the first electrode and the second electrode differ between the first mode and the second mode based on the load detected by the sensor.

2. The treatment system according to claim 1, wherein application of the high-frequency current to the grasped treatment target are different between the first mode and the second mode.

3. The treatment system according to claim 1, wherein a grasping force for grasping the treatment target between the first grasping piece and the second grasping piece differs between the first mode and the second mode.

4. The treatment system according to claim 1, wherein the control device is configured to determine whether the load detected by the sensor is less than a threshold.

5. The treatment system according to claim 4, wherein, when the load is greater than or equal to the threshold, the control device is configured to:
reduce the electric energy to the first electrode and the second electrode; and
increase a time for outputting the electric energy to be greater than a time for outputting electric energy when the load is less than the threshold.

6. The treatment system according to claim 4, wherein, when the load is greater than or equal to the threshold, the control device is configured to:
stop the output of the electric energy after starting the output of the electric energy to the first electrode and the second electrode; and
resume the output of the electric energy after stopping the output of the electric energy, such that the electric energy is output intermittently for a plurality of times.

7. The treatment system according to claim 4, wherein:
the control device includes an impedance detector configured to detect an impedance of a path supplying the electric energy to the first electrode and the second electrode, the path including the treatment target,
when the load is less than the threshold and the impedance reaches or exceeds a first impedance threshold, the control device is configured to stop the output of the electric energy to the first electrode and the second electrode; and
when the load is greater than or equal to the threshold and the impedance reaches or exceeds a second impedance threshold that is larger than the first impedance threshold, the control device is configured to stop the output of the electric energy.

8. The treatment system according to claim 4, wherein, when the load is greater than or equal to the threshold, the control device is configured to continue to stop the output of the electric energy to the first electrode and the second electrode.

9. The treatment system according to claim 4, wherein, when the load is greater than or equal to the threshold, the control device is configured to increase a grasping force for grasping the treatment target between the first grasping piece and the second grasping piece to be greater than a grasping force for grasping the treatment target between the first grasping piece and the second grasping piece when the load is smaller than the threshold.

10. A control device configured to be used with a treatment instrument, the treatment instrument comprising:
a first grasping piece including a first electrode;
a second grasping piece including a second electrode being different from the first electrode and the second grasping piece being configured to grasp a treatment target together with the first grasping piece by opening and closing with respect to the first grasping piece; and
a sensor, the sensor being configured to detect a load acting on the second grasping piece,
the control device being configured to:
obtain the load detected by the sensor;
output electric energy to the first electrode and the second electrode;
supply a high-frequency current to the treatment target for treating the treatment target; and
switch between a first mode and a second mode based on the load detected by the sensor, the second mode being different from the first mode in a control scheme such that outputs of the electric energy to the first electrode and the second electrode differ between the first mode and the second mode.

11. The control device according to claim 10, further configured to determine whether the load detected by the sensor is smaller than the threshold.

12. The control device according to claim 11, further configured to, when the load is greater than or equal to the threshold, increase a grasping force for grasping the treatment target between the first grasping piece and the second grasping piece to be greater than a grasping force for grasping the treatment target between the first grasping piece and the second grasping piece when the load is smaller than the threshold.

13. The treatment system according to claim 1, wherein when the load is greater than or equal to the threshold, the control device is configured to operate in the second mode.

14. The treatment system according to claim 1, wherein when the load is less than the threshold, the control device is configured to operate in the first mode.

15. The treatment system according to claim 1, wherein when the load is greater than or equal to the threshold, the control device is configured to adjust the electric energy output to the first electrode and the second electrode such that a blood vessel is sealed to a same degree as when the load is less than the threshold.
